(19) United States
(12) Patent Application Publication
(10) Pub. No.: US 2014/0180167 A1
(43) Pub. Date: Jun. 26, 2014

(54) SYSTEM AND METHOD FOR DETERMINING STRICTURE CHARACTERISTICS

(71) Applicant: The Trustees of the University of Pennsylvania, Philadelphia, PA (US)

(72) Inventors: Michael L. Kochman, Newtown, PA (US); Katherine J. Kuchenbecker, Philadelphia, PA (US)

(73) Assignee: The Trustees of the University of Pennsylvania, Philadelphia, PA (US)

(21) Appl. No.: 14/075,388

(22) Filed: Nov. 8, 2013

Related U.S. Application Data
(60) Provisional application No. 61/723,988, filed on Nov. 8, 2012.

(51) Int. Cl. A61B 5/107; A61M 29/00

(52) U.S. Cl. CPC A61B 5/1076; A61M 29/00
USPC 600/587

(57) ABSTRACT

Systems and methods for determining stricture characteristics are disclosed. A system for determining the characteristics of a stricture in a body lumen includes a dilation device, one or more sensors, and an output device. The dilation device is configured to be inserted in the body lumen at the location of the stricture. The one or more sensors are coupled to the dilation device for sensing characteristics of the dilation device during dilation. The output device is configured to indicate the characteristics of the dilation device. In order to determining the characteristics of the stricture, the dilation device may be inserted in the body lumen at the location of the stricture. The dilation device may then be inflated. Characteristics of the dilation device during inflation of the dilation device may then be sensed.

### CROSS REFERENCE TO RELATED APPLICATIONS

This application claims priority to U.S. Provisional Application Ser. No. 61/723,988, filed Nov. 8, 2012, which is incorporated herein, in its entirety, by reference.

### FIELD OF THE INVENTION

The present invention relates generally to the treatment of body lumens, and more particularly to systems and methods for determining characteristics of strictures in body lumens.

### BACKGROUND OF THE INVENTION

A stricture (or stenosis) is an abnormal narrowing in a body lumen. Strictures may arise from a variety of pathologic processes, which may be acquired, including iatrogenic, or congenital. Strictures may affect the gastrointestinal lumen, the biliary tract, the coronary vessels, and other vascular structures including the major blood vessels, such as the aorta and carotid arteries.

Various methods for treating such strictures have been proposed. Nevertheless, there remains a need for improved systems and methods related to the treatment of strictures in body lumens.

### SUMMARY OF THE INVENTION

Aspects of the present invention include systems and methods for determining stricture characteristics. According to one aspect of the present invention, a system for determining the characteristics of a stricture in a body lumen comprises a dilation device configured to be inserted in the body lumen at the location of the stricture; one or more sensors coupled to the dilation device for sensing one or more characteristics of the dilation device during dilation of the dilation device; and an output device configured to indicate the characteristics of the dilation device.

According to another aspect of the present invention, a method for determining the characteristics of a stricture in a body lumen comprises the steps of inserting a dilation device in the body lumen at the location of the stricture; inflating the dilation device; and sensing characteristics of the dilation device during inflation of the dilation device.

### BRIEF DESCRIPTION OF THE DRAWINGS

The invention is best understood from the following detailed description when read in connection with the accompanying drawing. Included in the drawing are the following figures:

FIG. 1 shows a schematic diagram of an exemplary embodiment of a system for determining the characteristics of a stricture in accordance with an aspect of the present invention; and

FIG. 2 shows a flow chart of an exemplary embodiment of a method for determining the characteristics of a stricture in accordance with an aspect of the present invention.

### DETAILED DESCRIPTION OF THE INVENTION

Although the invention is illustrated and described herein with reference to specific embodiments, the invention is not intended to be limited to the details shown. Rather, various modifications may be made in the details within the scope and range of equivalents of the claims and without departing from the invention.

Aspects of the present invention provide new systems and methods for determining the mechanical properties or characteristics of in vivo strictures. Although the invention may be described primarily with respect to esophageal strictures, it will be understood by one of ordinary skill in the art that the principles of the disclosed systems and methods will be applicable to the diagnosis of strictures in other body lumens, including cardiovascular lumens, for example.

Esophageal strictures may occur as a result of intrinsic issues (e.g. reflux scars from previous injury) or as a result of external factors (e.g. a tumor pressing onto the esophagus). Therapy selection is optionally driven by outcome studies, and the devices used to remediate strictures can be developed using empiric and iterative approaches.

Therapy for these various strictures optionally involves the use of surgical repair, dilation devices (e.g. balloons), and the placement of endoprosthetics (e.g. stents). For treatment with a dilation device, balloons are optionally inflated with handheld mechanical devices to a predetermined pressure.

To dilate an esophageal stricture, a medical treatment provider may optionally use a balloon catheter, a Savary dilator on a guidewire, or a Maloney dilator depending on the type and nature of the stricture. In general, a medical treatment provider may not know if a procedure to dilate a stricture has been successful. Thus, the results of treatment may be measured clinically, based on relief (or non-relief) of patient symptoms over time, or may be measured radiographically.

For example, a balloon device may be used to dilate a stricture. The balloon device may be inflated with a hand-pump operated by a medical treatment provider. Pressure of the balloon device may be increased to a pre-determined threshold. Then, the treatment provider may determine the success of the balloon device treatment clinically based on observation of the patient. Whether further sizes of balloons are used or whether an endoprosthetic is placed may then be determined at the discretion of the medical treatment provider. Alternatively, for treatment with an endoprosthetic placement, a stricture may be pre-dilated.

However, it has been discovered that data can be beneficially acquired from a dilation device. Thus, treatments can be selected or modified based on the measured characteristics or the nature of the stenosis.

The invention is best understood from the detailed description provided herein when read in connection with the accompanying drawing figures, which shows exemplary embodiments of the invention selected for illustrative purposes. The invention will be described with reference to the figures. Such figures are intended to be illustrative rather than limiting and are included herewith to facilitate the explanation of the present invention.

According to one aspect of the invention, and referring to the figures generally, a system 100 is provided for determining the characteristics of a stricture in a body lumen. The system 100 includes an inflatable dilation device 102 configured to be inserted in the body lumen at the location of the stricture. A pump 104 is coupled to the dilation device and configured to inflate the dilation device. One or more sensors 106 are coupled to the dilation device for sensing characteristics of the dilation device during inflation of the dilation device by the pump.

According to another aspect of the invention, and referring to the figures generally, a flowchart 200 is provided having steps for determining the characteristics of a stricture in a body lumen. The method includes inserting a dilation device in the body lumen at the location of the stricture (step 202), inflating the dilation device (step 204), and sensing characteristics of the dilation device during inflation of the dilation device (step 206).

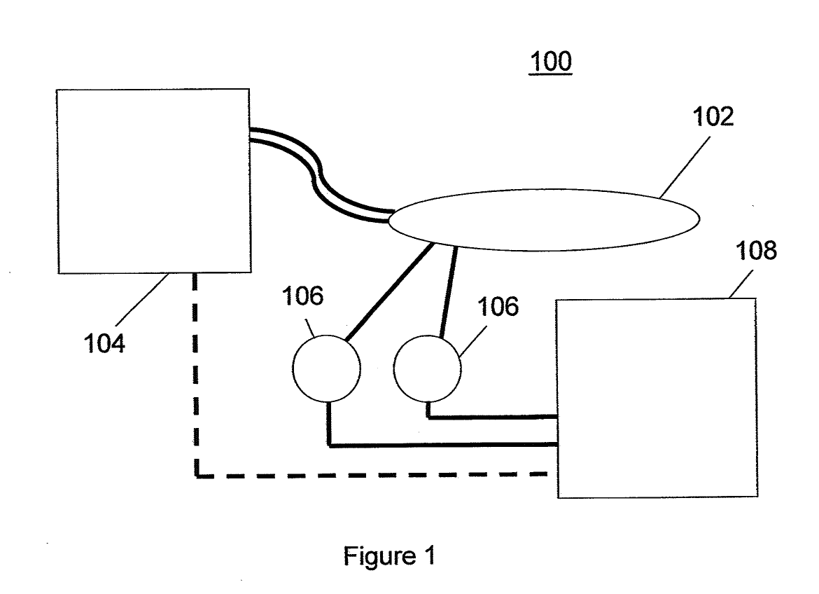

As an overview, FIG. 1 shows a block diagram of a system 100 for determining the characteristics of a stricture in accordance with an aspect of the present invention. Broadly, system 100 includes a dilation device 102, a pump 104, one or more sensors 106, and a data processing device 108. Additional details of system 100 will be described later.

Dilation device 102 is configured for insertion in a body lumen. In an exemplary embodiment, dilation device 102 is an inflatable dilation device for use in treating a stricture. Dilation device 102 may include a catheter for enabling inflation of dilation device 102 when inserted in a body lumen. Dilation device 102 may comprise a balloon, a balloon catheter, or other inflatable device that would be known to one of ordinary skill in the art in the treatment of strictures. Dilation device 102 may desirably be configured for insertion in any body lumen having a stricture, such as, for example, gastrointestinal luminal strictures and constrictions (e.g. strictures of the esophagus, pylorus, small bowel, colon, anastomoses, etc.), solid organ strictures and constrictions (e.g. strictures of the pancreas, liver, pancreatic duct, bile duct etc.), fallopian tube strictures and constrictions, constriction of cardiac valves, cardiac vessels, major blood vessels, and other vascular structures. Suitable dilation devices for insertion in the above body lumens will be understood to one of ordinary skill in the art from the description herein.

Pump 104 is configured to inflate an dilatable portion of dilation device 102. In an exemplary embodiment, pump 104 is an infusion pump having an inflation lumen coupled to an inflatable portion, such as a balloon, of dilation device 102. Pump 104 may provide an inflation fluid to dilation device 102 through a tube. The inflation fluid may inflate a dilation device which has been inserted in a body lumen at the location of a stricture. Pump 104 may desirably be controlled by a computer. For example, a computer may control the volume and/or pressure provided to dilation device 102 by pump 104.

An exemplary infusion pump for use with the present invention is the CT Injector computer-controlled infusion pump, provided by EZ-EM Inc. However, other suitable infusion pumps are contemplated. For example, suitable pumps 104 may include pumps that are typically used for controlled intravascular infusion of contrast for computed tomography studies and angiography. Other suitable infusion pumps for use with the present invention will be understood to one of ordinary skill in the art from the description herein.

One or more sensors 106 are coupled to one of dilation device 102 or pump 104 or both. In an exemplary embodiment, sensors 106 measure the characteristics of the dilation device 102 as it is dilated. Sensors 106 may desirably obtain continuous or nearly continuous readings or timed readings of the dilation device characteristics. Sensors 106 may be attached to a catheter or lumen of dilation device 102 such that the sensors 106 remain outside the patient's body when dilation device 102 is inserted. Alternatively, sensors 106 may be embedded within a catheter or lumen or directly within dilation device 102 such that sensors 106 are inserted within the patient along with dilation device 102. Sensors 106 may additionally be integrated with pump 104 for monitoring the inflation of dilation device 102.

As set forth above, sensors 106 measure the characteristics of the dilation device 102 as it is dilated. Sensors 106 particularly acquire information that is important for enabling system 100 to characterize the stricture, the intervention caused by dilation device 102, and the response of the stricture to the intervention. Such information includes, by way of example, pressure, volume, size, and shape/conformation of dilation device 102.

The one or more sensors 106 may desirably comprise at least one pressure transducer for measuring an internal pressure of dilation device 102 during inflation. The one or more sensors 106 may further desirably comprise at least one volume sensor for measuring the volume of dilation device 102 during inflation.

Other suitable sensors 106 include: volume flow rate sensors for monitoring the flow into and out of dilation device 102; force sensors coupled to a guidewire of dilation device 102 for monitoring how a medical treatment provider is pushing or pulling dilation device 102 during insertion and inflation; torque sensors coupled to a guidewire of dilation device 102 for monitoring how a medical treatment provider is twisting dilation device 102 during insertion and inflation; tactile pressure sensors coupled directly to dilation device 102 for measuring a pressure exerted on the surface of dilation device 102; pH sensors for monitoring the acidity surrounding dilation device 102; and temperature sensors for monitoring the temperature surrounding dilation device 102. One or more sensors 106 may also comprise fluoroscopic or similar imaging elements for determining the shape and size of dilation device 102 while inserted, for use in conjunction with imaging processes that would be known to one of ordinary skill in the art. The types and functions of sensors enumerated herein are illustrative and not limiting; other suitable sensors 106 for determining characteristics of the dilation device will be understood to one of ordinary skill in the art from the description herein.

Data processing device 108 is coupled to sensors 106. In an exemplary embodiment, data processing device 108 processes the data sensed by sensors 106. Data processing device 108 may be configured to capture data recorded by sensors 106. Data processing device 108 may further construct profiles for the stricture based on the data captured from sensors 106. Data processing device 108 may develop plots, graphs, or charts for presenting this information to a medical treatment provider.

For example, data processing device 108 may construct graphs showing the internal pressure of dilation device 102 over the time of inflation (time-pressure profiles), the volume of dilation device 102 over time of inflation (time-volume profiles), or the volume of dilation device 102 as it relates to the internal pressure of dilation device 102 (pressure-volume profiles). Data processing device 108 may also construct images showing the size or shape of dilation device 102 at certain times during inflation. It will be understood, however, that data processing device 108 may construct profiles, graphs, charts, or images from any measured characteristics based on the types of sensors 106 that are included in system 100.

Data processing device 108 is optionally integrated with pump 104 or with sensors 106. Data processing device 108 may further comprise a computer or any other suitable device for processing data measured by sensors 106. Data processing device 108 optionally includes a computer for controlling the inflation of dilation device 102 with pump 104, as described above. A suitable data processing device for use with system 100 will be understood by one of ordinary skill in the art from the description herein.

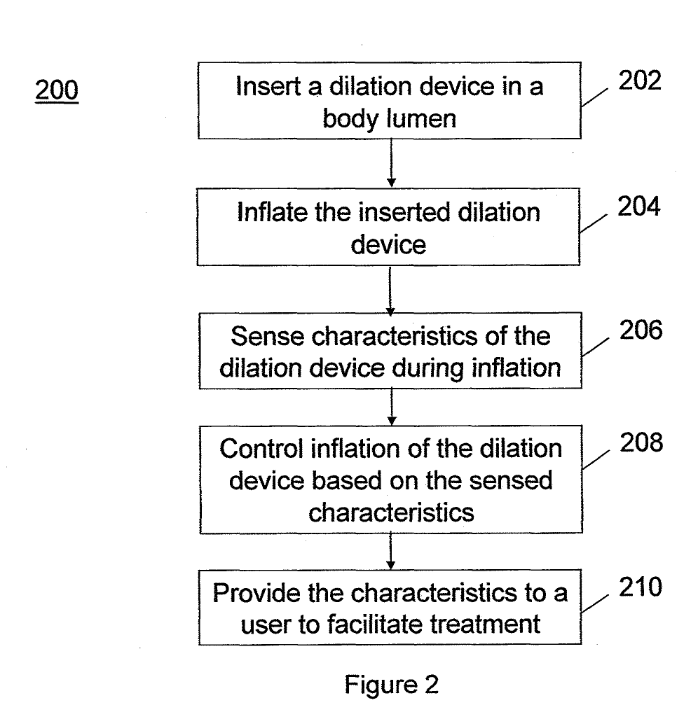

FIG. 2 shows a flow chart 200 of exemplary steps for determining the characteristics of a stricture in accordance with an aspect of the invention. To facilitate description, the steps of FIG. 2 are described with reference to the system components of FIG. 1. It will be understood by one of skill in the art from the description herein that one or more steps may be omitted and/or different components may be utilized without departing from the spirit and scope of the present invention.

In step 202, a dilation device is inserted in a body lumen. In an exemplary embodiment, dilation device 102 is inserted in a body lumen at the location of a stricture. Dilation device 102 may be inserted according to known methods, in accordance with the type of dilation device 102, the affected body lumen, and the location of the stricture. Insertion of a dilation device at the location of a stricture will be known to one of ordinary skill of the art from the description herein.

In step 204, the inserted dilation device is inflated. In an exemplary embodiment, pump 104 may be used to inflate dilation device 102. Pump 104 may inflate dilation device 102 by providing inflation fluid to dilation device 102 through an inflation lumen or tube. The inflation of dilation device 102 may desirably be controlled by a computer, such as where pump 104 is a computer-controlled infusion pump. For example, a computer may control the volume and/or pressure provided to dilation device 102 by pump 104.

In step 206, characteristics of the dilation device are sensed during inflation. In an exemplary embodiment, during inflation of dilation device 102 by pump 104, sensors 106 measure characteristics of dilation device 102. The characteristics may be sensed continuously or nearly continuously or at selected or predetermined time intervals. The characteristics measured by sensors 106 may desirably include the internal pressure of dilation device 102 and the volume of dilation device 102. However, as described above, any suitable sensors 106 may be used to measure characteristics of the dilation device, which will be understood by one of ordinary skill in the art.

In step 208, inflation of the dilation device is controlled based on the sensed characteristics of the stricture. In an exemplary embodiment, the characteristics of the dilation device measured by sensors 106 may be used to control the inflation of dilation device 102.

In step 210, the characteristics of the dilation device are provided to a user to facilitate treatment of the stricture. In an exemplary embodiment, data processing device 108 provides the sensed stricture characteristics to a medical treatment provider for diagnosis. As described above, data processing device 108 may be configured to capture data recorded by sensors 106. Data processing device 108 may further construct profiles for the stricture based on the data captured from sensors 106. Data processing device 108 may develop plots, graphs, or charts for presenting the profiles to a medical treatment provider.

The profiles generated by data processing device 108 may provide information about the stricture, and may be useful to a medical treatment provider in choosing a course of treatment of the stricture. For example, certain time-pressure profiles and time-volume profiles and pressure-volume profiles may correspond to specific types of strictures.

Additionally, certain profiles or stricture types may warrant a certain course of therapeutic treatment for the stricture. Stricture treatment options may include: attempting to widen a stricture in a bodily lumen; ceasing inflation of a dilation device at a target pressure; implementing one or several consecutive inflation actions, linked together directly or interleaved with deflations; pushing, pulling, or twisting a dilation device in situ to remediate problems with placement and/or aid in the determination of stricture characteristics; using a balloon to dilate a body lumen to facilitate placement of an endoprosthesis; and placement of an endoprosthesis and/or drug-delivery system. All of the above treatment options may be suggested or based on or informed by the profiles generated by data processing device 108.

Development of stricture profiles in accordance with aspects of the present invention may also enable medical treatment providers to immediately determine the outcome of a dilation procedure. Specifically, analysis of generated profiles and detected characteristics may enable medical treatment providers to determine: whether or not a tissue stricture/constriction was remediated; the degree of tissue injury; any change in the tissue and stricture compliance before and after inflation; the different between luminal remediation before and after inflation, and/or the short-term and/or long term needs for additional, repeated, or alternative therapies.

Additionally, should enough data collected regarding strictures in general, a medical treatment provider using the above invention may be able to generate therapeutic guidelines for the treatment of strictures based on observed stricture characteristics.

The above-described systems and methods provide a novel way to capture the characteristics of strictures that may lead to new knowledge. The above invention may allow for a scientific basis for the prediction of response to stricture therapy, allow for a more rational selection of therapy and better patient outcomes, lead to better general knowledge of stricture characteristics, lead to the development of electromechanical models of various strictures (for training and development purposes), and aid the development of devices, technologies, and techniques for the treatment of strictures. In short, it is believed that the above invention may allow for significant advances in medical care.

While preferred embodiments of the invention have been shown and described herein, it will be understood that such embodiments are provided by way of example only. Numerous variations, changes and substitutions will occur to those skilled in the art without departing from the spirit of the invention. Accordingly, it is intended that the appended claims cover all such variations as fall within the spirit and scope of the invention.

What is claimed:

1. A system for determining the characteristics of a stricture in a body lumen, comprising:
a dilation device configured to be inserted in the body lumen at the location of the stricture;
one or more sensors coupled to the dilation device for sensing one or more characteristics of the dilation device during dilation of the dilation device; and
an output device configured to indicate the characteristics of the dilation device.

2. The system of claim 1, wherein the dilation device is inflatable and comprises a balloon.

3. The system of claim 1, further comprising a pump coupled to the dilation device and configured to inflate the dilation device, wherein the pump comprises an infusion pump.

4. The system of claim 1, wherein at least one of the one or more sensors is a pressure transducer for monitoring an internal pressure of the dilation device.

5. The system of claim 1, wherein at least one of the one or more sensors is a volume sensor for monitoring a volume of the dilation device.

6. The system of claim 1, further comprising a profile generator configured to generate a dilation profile including at least one characteristic of the dilation device.

7. The system of claim 6, the profile generator being configured to generate at least one of a time-pressure dilation profile, a time-volume dilation profile, and a volume-pressure dilation profile.

8. A method for determining the characteristics of a stricture in a body lumen, comprising the steps of:
inserting a dilation device in the body lumen at the location of the stricture;
inflating the dilation device; and
sensing characteristics of the dilation device during inflation of the dilation device.

9. The method of claim 8, further comprising the step of:
controlling the inflation of the dilation device based on the characteristics of the dilation device.

10. The method of claim 8, further comprising the step of:
providing the characteristics of the dilation device to a user to facilitate treatment of the stricture.

11. The method of claim 10, wherein the providing step comprises:
generating a profile for the stricture based on the sensed characteristics of the dilation device; and
providing the profile for the stricture to the user.

12. The method of claim 8, wherein the characteristics of the dilation device include the internal pressure of the dilation device.

13. The method of claim 8, wherein the characteristics of the dilation device include the volume of the dilation device.

14. A method for treating a stricture in a body lumen, the stricture being dilated with a dilation device, the method comprising the steps of:
sensing characteristics of the dilation device during inflation of the dilation device at the location of the stricture;
generating a profile for the stricture based on the sensed characteristics of the dilation device; and
treating the stricture based on the profile for the stricture.
